(19) United States
(12) Patent Application Publication
(10) Pub. No.: US 2022/0338902 A1
(43) Pub. Date: Oct. 27, 2022

(54) FLEXIBLE NEEDLE

(71) Applicant: UNOMEDICAL A/S, Lejre (DK)

(72) Inventor: Erik Othel-Jacobsen, Snekkersten (DK)

(21) Appl. No.: 17/727,008

(22) Filed: Apr. 22, 2022

Related U.S. Application Data
(63) Continuation of application No. PCT/EP2022/060653, filed on Apr. 22, 2022.
(60) Provisional application No. 63/178,802, filed on Apr. 23, 2021.

(51) Int. Cl. A61B 17/34; A61L 31/02

(52) U.S. Cl. CPC A61B 17/3478; A61L 31/022; A61B 2017/00867

(57) ABSTRACT

An exemplary needle defines a lumen and includes a proximal portion and a distal portion. The proximal portion defines a first port of the lumen, the distal portion defines at least one second port of the lumen, and the lumen extends between and connects the first port and the at least one second port. The distal portion also defines a tip portion configured for insertion into patient skin. In certain embodiments, the distal portion includes a compressible region including at least one slit. In certain embodiments, the distal portion includes an opening and a ramp configured to urge patient tissue within the distal portion toward the opening during insertion of the distal portion into patient tissue. In certain embodiments, the needle is formed of nitinol.

### CROSS-REFERENCE TO RELATED DISCLOSURES

The present disclosure is a continuation of International Application No. PCT/EP2022/060653 filed on Apr. 22, 2022 and claims the benefit of U.S. Provisional Application No. 63/178,802 the contents of which are hereby incorporated herein in entirety.

### TECHNICAL FIELD

The present disclosure generally relates to needles, and more particularly but not exclusively relates to flexible needles configured for use in infusion devices.

### BACKGROUND

Many current infusion devices utilize a soft cannula, insertion of which into the patient's tissue requires a rigid needle. Insertion needles can add to the pain and tissue trauma associated with insertion due to size differences between the needle and the cannula. More particularly, by having a soft cannula around the needle, the wound created by the needle is too small for the soft cannula. The cannula therefore typically needs to be pressed through the smaller hole, which will cause more pain to the patient. Moreover, many soft cannulas do not sufficiently resist kinking, which can stop the flow of the therapeutic agent. On the other hand, if the infusion is performed with a conventional needle, the tip of the rigid needle will engage the adjacent tissue during patient movement, which may worsen the wound and cause additional trauma to the patient. For these reasons among others, there remains a need for further improvements in this technological field.

### SUMMARY

An exemplary needle defines a lumen and includes a proximal portion and a distal portion. The proximal portion defines a first port of the lumen, the distal portion defines at least one second port of the lumen, and the lumen extends between and connects the first port and the at least one second port. The distal portion also defines a tip portion configured for insertion into patient skin. In certain embodiments, the distal portion includes a compressible region including at least one slit. In certain embodiments, the distal portion includes an opening and a ramp configured to urge patient tissue within the distal portion toward the opening during insertion of the distal portion into patient tissue. In certain embodiments, the needle is formed of nitinol. Further embodiments, forms, features, and aspects of the present application shall become apparent from the description and figures provided herewith.

### BRIEF DESCRIPTION OF THE FIGURES

FIG. 1 is a first perspective view of a flexible needle according to certain embodiments.

FIG. 2 is a second perspective view of the flexible needle.

FIG. 3 illustrates a distal portion of the flexible needle with a compressible region in an expanded state.

FIG. 4 illustrates the distal portion of the flexible needle with the compressible region in a compressed state.

FIG. 5 is a perspective view of a tip portion of the flexible needle.

FIG. 6 is a cutaway view of the tip portion of the flexible needle.

FIG. 7 is a graph illustrating certain properties of nitinol.

FIG. 8 is a stress-strain diagram comparing certain properties of the illustrated flexible needle with those of a conventional needle.

FIG. 9 is a graph illustrating martensite start temperatures versus nickel/titanium ratio for nitinol.

FIG. 10 illustrates a distal portion of a needle according to certain embodiments.

FIG. 11 is a graph illustrating certain properties of needles according to certain embodiments in comparison with a conventional cannula.

### DETAILED DESCRIPTION OF ILLUSTRATIVE EMBODIMENTS

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Items listed in the form of “A, B, and/or C” can also mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

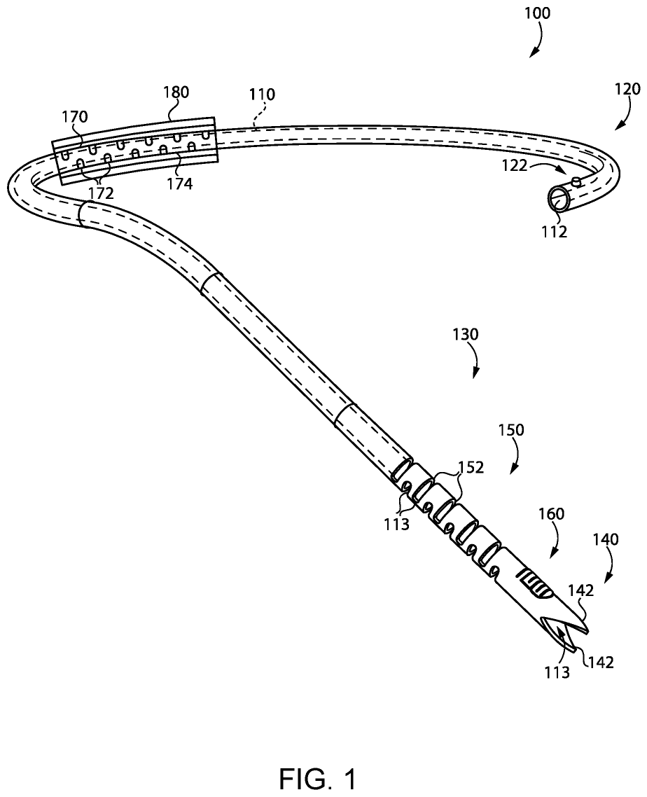

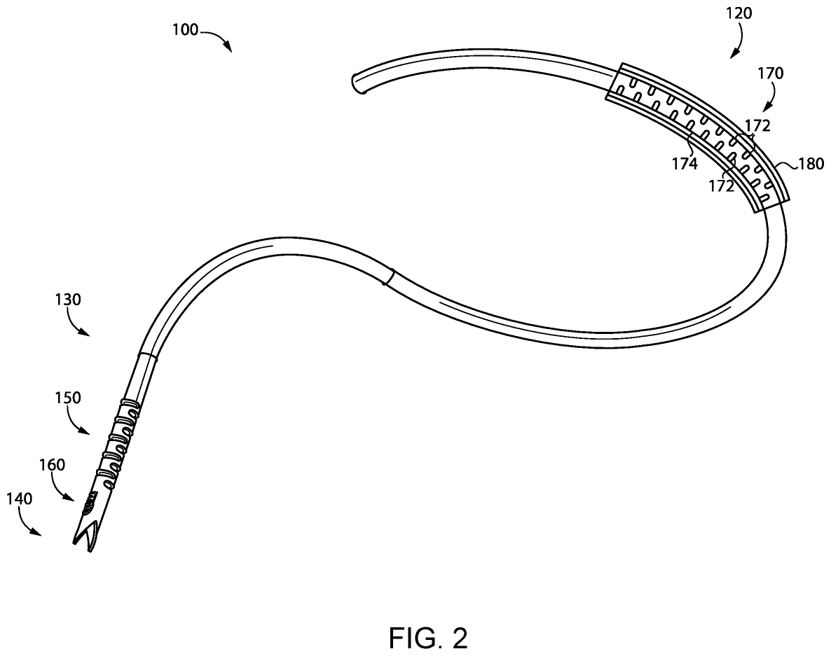

With reference to FIGS. 1 and 2, illustrated therein is a flexible needle 100 according to certain embodiments. The needle 100 defines a lumen 110, and generally includes a proximal portion 120 and a distal portion 130. The distal portion 130 includes a tip portion 140, and may further comprise one or both of a distal compressible region 150 and/or a tab 160. In certain forms, the proximal portion 120 includes a proximal compressible region 170 and a sheath 180 that covers the compressible region 170. In the illustrated form, the needle 100 is configured for use with an infusion device in which the proximal portion 120 is mounted within the infusion device and the infusion device is operable to insert the distal portion 130 into patient tissue.

The needle 100 has an outer surface 102 and an inner surface 104, the inner surface 104 at least partially defining the lumen 110. It has been found that it may be desirable for the outer surface 102 and the inner surface 104 to have different hydrophobic/hydrophilic characteristics. More particularly, it has been found that it may be desirable for the outer surface 102 to be more hydrophilic while having the inner surface 104 be more hydrophobic. A relatively hydrophilic outer surface 102 may aid adhesion to tissue, and it has been found that human tissue is more prone to accept the presence of an inert foreign body if the surface thereof is hydrophilic. A relatively hydrophobic inner surface 104 may aid in dispersion of therapeutic agent by discouraging the therapeutic agent from adhering to the inner surface 104. In certain embodiments, one or both of the outer surface 102 and/or the inner surface 104 may comprise a surface treatment that alters the hydrophobicity/hydrophilicity of the surface. Such surface treatments may, for example, involve the use of chemical treatments, electrochemical treatments, polishing, diamonds, etching, and/or coating.

In certain embodiments, such as those in which the needle 100 is formed of nitinol, the at least one surface treatment may involve exposing the outer surface 102 to ultraviolet light while the inner surface 104 remains unexposed to the ultraviolet light. Such a treatment may reduce the contact angle of the outer surface 102 to a relatively low contact angle (e.g., about 20°)while the contact angle for the inner surface 104 remains relatively high (e.g., about 80°).

The lumen 110 includes first port 112 defined by the proximal portion 120 and at least one second port 113 defined by the distal portion 130. In use, a therapeutic agent (e.g., insulin or a chemotherapy drug) may be provided to the lumen 110 via the first port 112 and flow through the lumen 110 for discharge into patient tissue via the at least one second port 113. As such, the first port 112 may be referred to herein as the inlet 112, and the second port(s) 113 may be referred to as the outlet(s) 113. While the needle 100 is described with specific reference herein to infusion devices, it should be appreciated that the needle 100 may be utilized to withdraw fluid from patient tissue. In such a case, the first port 112 may be an outlet and the one or more second ports 113 may be inlets. As described herein, the illustrated needle 100 comprises a plurality of second ports 113, which may facilitate the distribution of the therapeutic agent into the patient tissue.

In the illustrated form, the proximal portion 120 is configured to be mounted within the infusion device and to provide sufficient rigidity that forces exerted on the proximal portion 120 can be utilized to drive the distal portion 130 into patient tissue. In certain forms, the proximal portion 120 may include a mounting feature 122 that facilitates coupling of the proximal portion 120 to the infusion device, and which may orient the needle 100 in a specific orientation to encourage the tip portion 140 to enter the patient tissue in an appropriate orientation. As described herein, the illustrated proximal portion 120 also includes a compressible region 170 comprising a plurality of slits 172, and a sheath 180 covers the compressible region 170 to prevent fluid from leaking via the slits 172. It is also contemplated that the compressible region 170 and the sheath 180 may be omitted in certain embodiments.

The distal portion 130 extends along a distal portion longitudinal axis 131, is configured for insertion into patient tissue, and includes a tip portion 140 that facilitates such insertion as described herein. The distal portion 130 may further comprise one or both of a compressible region 150 and/or a tab 160, the functions of which are described herein.

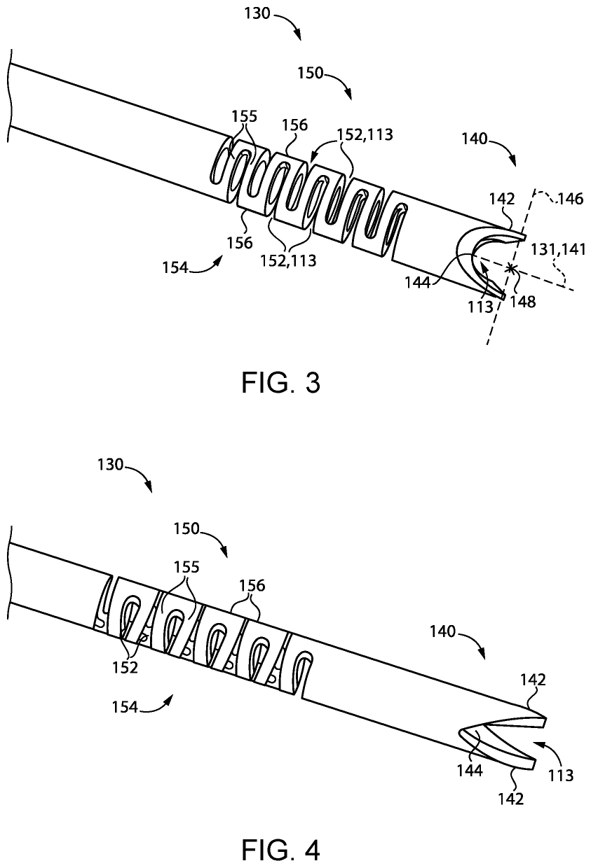

With additional reference to FIG. 3, the tip portion 140 extends along a tip portion longitudinal axis 141, includes at least one sharpened tip 142 that facilitates insertion into patient tissue, and in the illustrated form includes a plurality of sharpened tips 142 that are spaced from one another by recesses 144. More particularly, the illustrated tip portion 140 includes a pair of sharpened tips 142 that are positioned diametrically opposite one another relative to the tip portion longitudinal axis 141 such that a first transverse axis 146 extends between the tips 142 and a second transverse axis 148 extends between the recesses 144. The first transverse axis 146 and the second transverse axis 148 are transverse to one another and to the longitudinal axis 141, and in the illustrated form, the axes 141, 146, 148 are mutually orthogonal.

While the illustrated tip portion 140 includes a pair of tips 142, it is also contemplated that the tip portion 140 may include more sharpened tips 142. For example, the tip portion 140 may include three sharpened tips spaced about 120° from one another relative to the longitudinal axis 141. In the illustrated form, the tip portion 140 is an open tip portion that defines at least one of the second port(s) 113. It is also contemplated that the tip portion 140 may be a closed tip portion that does not define a port of the lumen 110, for example in embodiments in which one or more second ports 113 are formed in the compressible region 150.

As noted above, the illustrated tip portion 140 includes a plurality of sharpened tips 142. In other embodiments, the tip portion 140 may include a single sharpened tip 142. However, it has been found that providing multiple tips 142 may provide the needle 100 with greater stability during insertion. For example, in embodiments with a single tip 142, insertion of the tip portion 140 may result in the generation of forces transverse to the insertion axis, which may cause bending of the needle 100 from its desired insertion angle. However, if multiple tips 142 are provided such that the tips 142 taper to recesses 144, the generation of such bending moments may be reduced or prevented.

With additional reference to FIG. 4, the compressible region 150 includes at least one slit 152, each slit 152 defining a corresponding one of the second port(s) 113. In the illustrated form, the compressible region 150 comprises a plurality of discrete slits 152 such that the remaining material forms a serpentine structure 154. More particularly, the slits 152 extend transverse to the distal portion longitudinal axis 131, and in the illustrated form extend substantially perpendicular to the distal portion longitudinal axis 131. In addition or as an alternative to extending transverse to the longitudinal axis 131, one or more slits 152 may extend in a direction of the longitudinal axis 131. As one example, the compressible region 150 may include one or more helical slits.

As noted above, the illustrated compressible region 150 includes a serpentine structure 154 that is defined at least in part by the plurality of slits 152. The serpentine structure 154 includes transverse sections 155 that extend transverse to the longitudinal axis 131 and longitudinal connecting portions 156 that extend in a direction of the longitudinal axis 131. In the illustrated form, the connecting portions 156 are positioned on opposite sides of the longitudinal axis 131 such that the compressible region 150 provides greater flexibility in one direction transverse to the longitudinal axis 131 than in another direction transverse to the longitudinal axis 131. While other configurations are contemplated, the illustrated connecting portions 156 are aligned with the tip portions 142 such that the compressible region 150 is more flexible in directions defined by the first transverse axis 146 (i.e., the axis extending between the tips 142) and is less flexible in directions defined by the second transverse axis 148 (i.e., the axis extending between the recesses 144). Such selective flexibility may aid in guiding the distal portion 130 during insertion into patient tissue.

In the illustrated form, the slits 152 are positioned to provide the compressible region 150 with varying degrees of flexibility in different directions transverse to the longitudinal axis 131. It is also contemplated that the slits 152 may be positioned to provide uniform flexibility in different directions transverse to the longitudinal axis 131. For example, an array of ten slits spaced evenly about the longitudinal axis 131 may provide for even flexibility in all directions transverse to the longitudinal axis 131. However, by providing for less flexibility along a particular direction, the insertion of the needle 100 may be facilitated as described above.

The compressible region 150 of the distal portion 130 is operable to transition between an expanded state (FIG. 3) and a compressed state (FIG. 4). More particularly, during insertion of the distal portion 130 into patient tissue, the serpentine structure 154 at least partially collapses, thereby at least partially closing one or more of the slits 152 as the compressible region 150 adopts the compressed state. When so compressed, the rigidity of the compressible region 150 increases, thereby facilitating further insertion of the distal portion 130 into the tissue. When the inserting force is removed and/or reversed, the compressible region 150 returns to its expanded state, thereby at least partially opening the slits 152 and the second ports 113 defined thereby. As a result, the therapeutic agent is operable to flow through the ports 113 defined by the slits 152, thereby increasing the area for diffusion of the therapeutic agent into patient tissue.

In addition to facilitating the dispersion of therapeutic agent into patient tissue, the compressible region 150 may aid in discouraging the tip portion 140 from causing excessive damage to patient tissue. When a conventional rigid needle is inserted into patient tissue, various types of forces (e.g., movement of the patient or the device attached to the needle) may cause the tip of the rigid needle to dig into virgin tissue and/or scrape surrounding tissue. However, the compressible region 150 is capable of at least partially absorbing these forces to thereby discourage the sharpened tip(s) 142 from causing further damage to patient tissue.

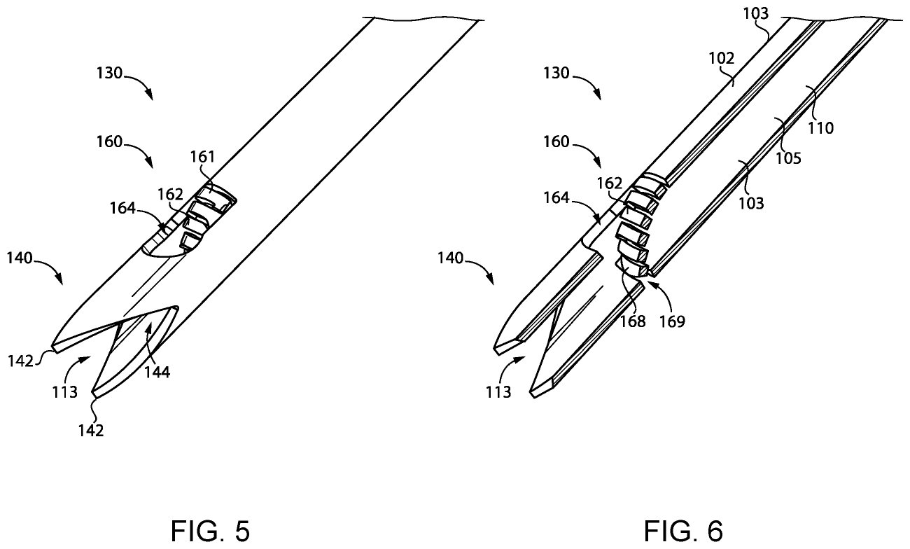

With additional reference to FIGS. 5 and 6, the illustrated tab 160 is cut from the outer wall of the distal portion 130 such that a proximal end 161 of the tab 160 remains connected with the remainder of the distal portion 130. The tab 160 is deformed into the interior of the needle 100 to define a ramp 162 that at least partially obstructs the lumen 110 and which leads to an opening 164 defined in the space once occupied by the tab 160. In certain forms, the tab 160 may include one or more slits 166 that increase the flexibility of the tab 160 and/or facilitate the flow of therapeutic agent through the ramp 164. Additionally or alternatively, a distal tip 168 of the tab 160 may be received in a slot 169 formed opposite the opening 164 to at least selectively retain the tab 160 in its angled position.

During insertion of the tip portion 140 into patient tissue, it may be the case that a plug of patient tissue enters the port 113 defined by the tip portion 140 in a manner similar to that which occurs during a traditional biopsy procedure. Those skilled in the art will readily recognize that such a tissue plug, particularly one comprising dermal tissue, may obstruct the lumen 110 and discourage the flow of therapeutic agent into patient tissue. The tab 160 may aid in clearing such plugs by causing the dermal tissue to ride along the ramp 162 during insertion of the distal portion 130 into patient tissue such that the dermal plug exits the lumen 110 via the opening 164. While the illustrated tab 160 is provided in a flexible needle 100, it is also contemplated that a ramp and opening such as those described in connection with the tab 160 may find use in other forms of needles, such as rigid needles.

The proximal compressible region 170 is substantially similar to the distal compressible region 150, and generally includes one or more slits 172 that enable the proximal compressible region 170 to expand and compress in a manner similar to that described above with reference to the distal compressible region 150. Thus, like the distal compressible region 150, the proximal compressible region 170 may absorb forces to aid in discouraging the tip portion 140 from causing further damage to patient tissue.

Those skilled in the art will readily recognize that the slits 172 of the proximal compressible region 170 may provide an avenue by which therapeutic agent may exit the portion of the needle 100 that is within the infusion device, which would generally be undesirable. In order to discourage such leakage, the proximal portion 120 may be provided with a sheath 180 that covers the proximal compressible region 170 to prevent fluid from exiting the slit(s) 172 of the compressible region 170. The sheath 180 may be provided in a material suitable for sealing the compressible region 170, such as a soft polymer, polyurethane, or elastomer.

It should be evident from the foregoing that the compressible region(s) 150, 170, may provide the needle 100 with one or more advantages. For example, the compressible region(s) 150/170 may stack up during insertion and thereby form a rigid, pushable and compact structure, as illustrated in FIG. 4. Once the needle 100 is in place in the desired position in the tissue, the force may be removed, either by built-in expansion forces generated by the serpentine structure 154/174, or by a small active backwards unloading of the needle 100. For the distal compressible region 150, this allows the slits 152 to open and act as array of ports or outlets 113 and act as a pathway for the therapeutic agent to reach the patient tissue over a large contact area. Additionally, the compressible region(s) 150, 170 may act as damping mechanisms that delay or absorb the motions imposed by movement of the infusion device, thereby preventing or reducing the unwanted movements of the sharp needle tip(s) 142 in the tissue during treatment.

As noted above, the illustrated needle 100 is a flexible needle, and is flexible in at least the compressible region(s) 150, 170. In certain embodiments, the needle 100 may also be flexible in regions other than the compressible region(s) 150, 170. In order to provide such flexibility, the needle 100 may, for example, be formed of a superelastic material. In the illustrated form, the needle 100 is formed of nitinol that is at least primarily austenitic at body temperature (about 37° C.).

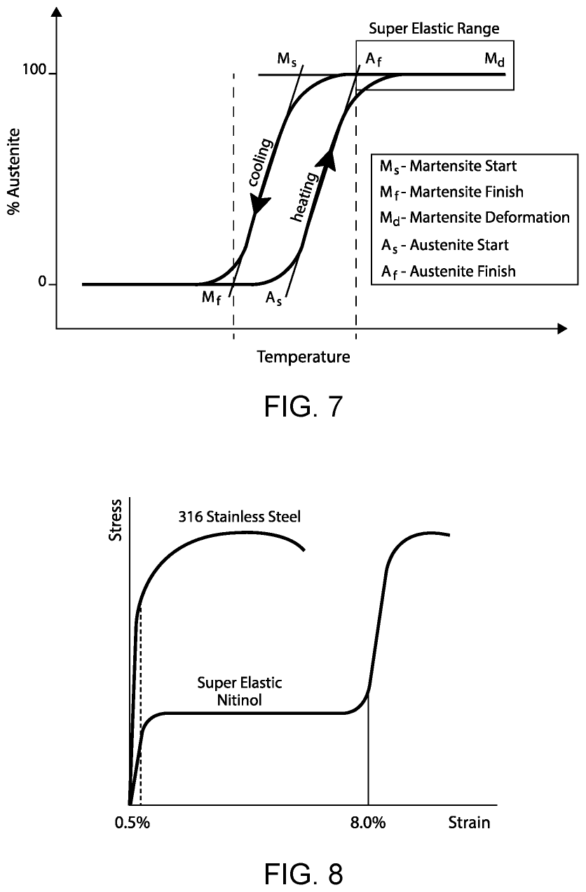

With additional reference to FIG. 7, illustrated therein is a graph demonstrating the behavior of nitinol during heating and cooling thereof. At relatively lower temperatures, the nitinol is fully martensitic. Heating of the nitinol beyond its austenite start temperature Aₛ causes the nitinol to transition to the austenitic phase, with the nitinol being fully austenite at temperatures above its austenite finish temperature A_{f}. From the fully austenitic phase, cooling of the nitinol beyond its martensite start temperature Mₛ causes the nitinol to transition to the martensitic phase, with the nitinol being fully martensitic at temperatures below its martensite finish temperature M_{f}.

One property of nitinol that may be advantageous for certain implementations of the needle 100 is the superelasticity of austenitic nitinol. More particularly, when the nitinol is substantially fully austenitic (e.g., greater than 95% austenitic), nitinol exhibits a relatively large superelastic range. This superelasticity enables the nitinol to elastically deform through a relatively broad range of stresses, which in turn enables a needle 100 formed of nitinol to elastically bend without causing permanent deformation of the needle 100.

With additional reference to FIG. 8, illustrated therein is a stress-strain diagram comparing the behavior of a needle formed of 316 stainless steel with a needle 100 formed of nitinol. As is evident from the figure, the nitinol needle 100 has a large range of elastic deformation in which increased strain does not significantly increase the stresses within the needle. In the illustrated form, stresses begin to rise at about 8% strain. As such, it may be advisable to maintain the strain associated with the needle 100 below about 6% in order to ensure that the needle 100 remains within its elastic deformation range.

From the foregoing, it should be evident that a needle 100 formed of superelastic nitinol may exhibit greater flexibility than a similarly-configured needle formed of a more conventional material, such as stainless steel. Those skilled in the art will also readily appreciate that the range of superelasticity for a particular formulation of nitinol depends largely upon its nickel/titanium ratio. As described herein, certain embodiments of the needle 100 may be formed of a nitinol alloy with a nickel/titanium ratio specifically selected to provide the needle 100 with particular properties.

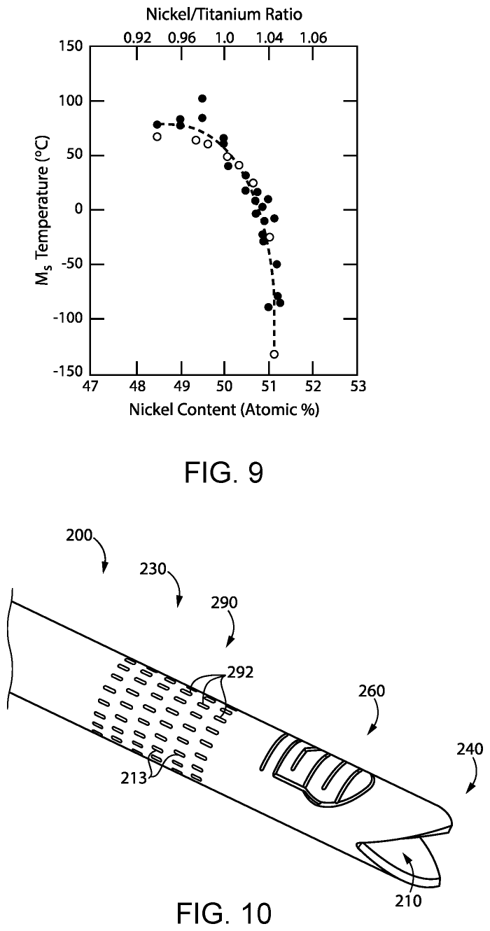

As noted above, a reduction in the austenite percentage of nitinol can occur when the nitinol is cooled below its martensite start temperature Mₛ, which is correlated with the nickel/titanium ratio of the nitinol. This correlation is illustrated in FIG. 9. Armed with this knowledge, those skilled in the art will readily appreciate that a particular martensite start temperature Mₛ can be achieved by providing the nitinol with an appropriately-selected nickel/titanium ratio.

As noted above, certain embodiments of the needle 100 are specifically configured for use with infusion devices. Those skilled in the art will readily recognize that such infusion devices can be worn by the patient such that at least part of the distal portion 130 remains embedded in the patient's soft tissue. Due to the superelastic properties of austenitic nitinol and/or the provision of one or more compressible regions 150, 170, the needle 100 may reduce tissue damage by remaining flexible as described above. However, should the martensite percentage of the distal portion 130 begin to rise, the distal portion 130 will be exposed to stress and perform a plastic deformation, resulting in an irregular, uncontrolled shape change. This may lead to tissue damage for reasons analogous to those set forth above with regard to rigid needles. While the patient's body heat may aid in maintaining the nitinol in its austenitic phase, certain embodiments of the needle 100 may be embedded in tissue to a relatively low insertion depth, such as about 3 mm to about 4 mm. As such, cold temperatures experienced by the patient may cause the nitinol to transition to its martensitic phase if the martensite start temperature Mₛ is too high. Thus, in certain embodiments, the needle 100 may be formed of a nitinol alloy selected to remain substantially fully austenitic (e.g., at least 95% austenitic) in temperatures likely to be experienced by the patient in order to reduce the risk of the needle 100 becoming martensitic.

In certain embodiments, the nitinol may be selected such that the nitinol exhibits selected characteristics with reference to a particular temperature. In certain embodiments, the particular temperature may be a temperature of 10° Celsius or lower, a temperature of 0° Celsius or lower, a temperature of −10° Celsius or lower, and/or a temperature of −20° Celsius or lower. In certain embodiments, the nitinol may be configured to remain at least 95% austenitic at the particular temperature. In certain embodiments, the nitinol may configured to remain at least 95% austenitic when cooled from a temperature at which the nitinol is fully austenitic to the particular temperature. In certain embodiments, the martensite start temperature may be the particular temperature. In certain embodiments, the nickel/titanium ratio of the nitinol may be selected to provide the nitinol with one or more selected characteristics. In certain embodiments, an atomic ratio of nickel to titanium for the nitinol is between 1.01 and 1.05. In certain embodiments, an atomic ratio of nickel to titanium for the nitinol is between 1.02 and 1.04.

The superelasticity of the needle 100 may facilitate its use in an infusion device, among other uses. For example, due to the superelasticity and flexibility provided by the concepts described herein, the needle 100 may be capable of changing its geometry from a circular form (e.g., for storage in the infusion device) to a straight line (e.g., for insertion into patient tissue) and vice versa by loading or unloading of forces onto the needle 100. This allows the needle 100 to coil up into the housing of the infusion device for transportation either onward to insertion into the soft tissue or after use to coil back into the housing. This feature may enable the needle 100 to require minimal volumetric space occupancy during transport or storage, and may further enable the needle 100 to form a straight line momentarily as it leaves the housing and enters the soft tissue, while coiling during retraction after use. This may aid in ensuring that the patient never sees the needle 100, and may prevent the healthcare provider from being exposed to a sharp, unprotected needle. In certain forms, the inserter device may be configured such that the strain undergone by the needle 100 during insertion and retraction remains less than 6%.

With additional reference to FIG. 10, illustrated therein is a distal portion 230 of a needle 200 according to certain embodiments. The needle 200 is substantially similar to the above-described needle 100, and similar reference characters are used to illustrate similar elements and features. For example, the needle 200 includes a lumen 210 corresponding to the above-described lumen 110, and the distal portion 230 includes a tip portion 240 and a tab 260 corresponding to the above-described tip portion 140 and tab 160. The proximal end of the needle 200 may be substantially similar to the proximal end 120 of the above-described needle 100. In the interest of conciseness, the following description of the needle 200 focuses primarily on features that differ from those described with reference to the needle 100.

In place of the flexible region 150, the needle 200 includes a dispersion region 290 comprising a plurality of apertures 292, each of which defines a corresponding port 213 of the lumen 210. The dispersion region 290 extends about at least a portion of the circumference of the needle 200, and also extends longitudinally. While the dispersion region 290 does not necessarily aid in providing the distal portion 230 with flexibility beyond that provided by the properties of the material of which the needle 200 is formed (e.g., nitinol), the dispersion region 290 may nonetheless aid in the dispersion of therapeutic agent in a manner analogous to that described above with reference to the compressible region 150.

The apertures 292 can be designed for aiding the flow of therapeutic agents with different viscosities. In certain embodiments, the apertures 292 may be designed as longitudinal slits, circular holes, arrays of holes or slits, and openings in increasing size or decreasing size to equalize drug pressure over a larger distance and surface of the needle 200. In certain embodiments, the dispersion region 290 may be tailored to vary according to tissue type and/or pressure, and/or may be structured for better stability and/or to maximize flow at specific viscosities.

As noted above, at least some embodiments of the needles 100, 200 described herein are flexible. Certain embodiments of the needles described herein are capable of flexing by at least 90° along their length without exhibiting significant plastic deformation. Certain embodiments of the needles described herein are capable of coiling without exhibiting significant plastic deformation. Certain embodiments of the needles described herein exhibit superelasticity during normal use. Certain embodiments of the needles described herein exhibit superelasticity at temperatures between a lower threshold and an upper threshold. In certain embodiments, the upper threshold is at least 40° C., at least 45° C., or at least 50° C. In certain embodiments, the lower threshold is 10° C. or less, 0° C. or less, −10° C. or less, or −20° C. or less.

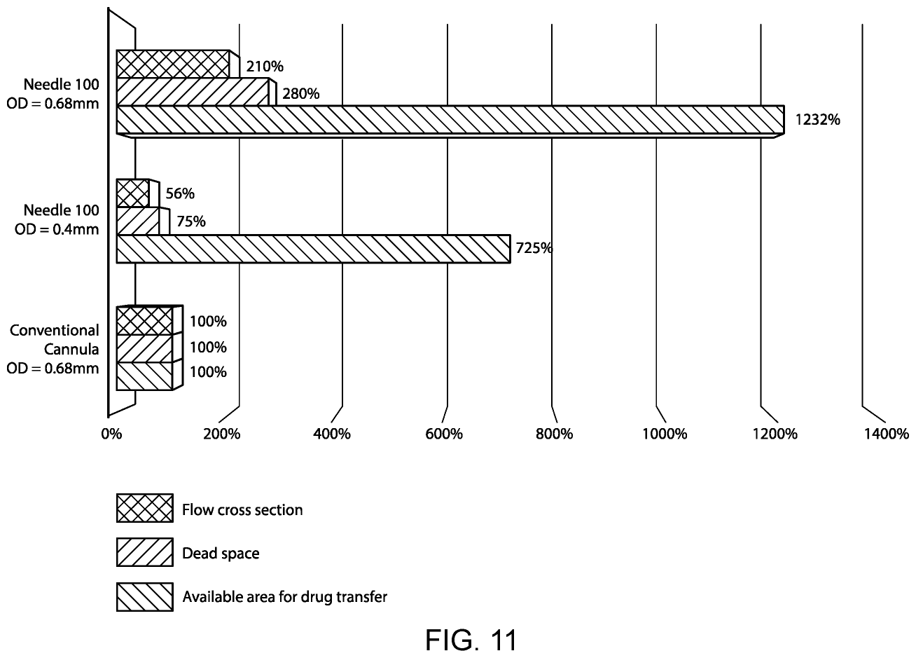

With additional reference to FIG. 11, provided therein is a graph demonstrating certain properties of exemplary embodiments of the needle 100 in comparison to a conventional cannula. More particularly, FIG. 11 illustrates the flow cross-section, dead space, and drug transfer area provided by two embodiments of the needle 100 in comparison to a 100% benchmark provided by a conventional cannula having an outer diameter (OD) of 0.68 mm.

The uppermost set of bars demonstrates the properties for a needle 100 having the same OD of 0.68 mm. Such larger needles 100 and cannulas may, for example, be utilized in infusion devices for non-diabetic use (e.g., for use in chemotherapy infusion devices). Due to the lesser thickness of the walls of the needle 100 in comparison to those of the conventional cannula, the needle 100 provides more than twice the flow cross-section in comparison to the conventional cannula, which may facilitate flow of the therapeutic agent. While this is correlated with an increase in dead space for the needle 100, the drug transfer area provided by the needle 100 is over twelve times that of the conventional cannula, which may result in improved drug diffusion characteristics.

The middle set of bars demonstrates that for a needle 100 having a smaller OD of 0.4 mm. Such a smaller needle 100 may, for example, be utilized in infusion devices for diabetic use (e.g., for use in insulin infusion devices). While the flow cross-section is just over half that of the conventional cannula, the dead space is reduced by about 25%. Additionally, the drug transfer area provided by the smaller-diameter needle 100 is over seven times that of the conventional cannula, which may result in improved drug diffusion characteristics.

Certain embodiments of the present application relate to a flexible needle 100 defining a lumen 110, the flexible needle 100 comprising: a proximal portion 120 defining a first port 112 of the lumen 110; and a distal portion 130 defining at least one second port 113 of the lumen 110, wherein the lumen 110 extends between and connects the first port 112 and the at least one second port 113, and wherein the distal portion 130 comprises: a tip portion 140 comprising at least one sharpened tip 142 configured to facilitate insertion of the distal portion 130 into patient tissue; and a compressible region 150 comprising at least one slit 152, each slit 152 defining a corresponding second port 113 of the at least one second port 113; wherein the compressible region 150 is configured to compress during insertion of the distal portion 130 into patient tissue to thereby close one or more second ports 113 of the at least one second port 113, and to thereafter expand to open the one or more second ports 113.

In certain embodiments, one or more slits 152 of the at least one slit 152 extends transverse to a longitudinal axis 131 of the distal portion 130.

In certain embodiments, the at least one second port 113 comprises a plurality of second ports 113; and wherein the tip portion 140 further defines an additional second port 113 of the plurality of second ports 113.

In certain embodiments, the at least one sharpened tip 142 comprises a plurality of sharpened tips 142.

In certain embodiments, the plurality of sharpened tips 142 are evenly spaced about a longitudinal axis 141 of the tip portion 140.

In certain embodiments, the compressible region 150 is more flexible in a first direction transverse to a longitudinal axis 131 of the distal portion 130 and is less flexible in a second direction transverse to the longitudinal axis 131 of the distal portion 130.

In certain embodiments, the proximal portion 120 further comprises: a second compressible region 170 comprising at least one second slit 172; and a sheath 180 covering the second compressible region 170 to prevent fluid from exiting the at least one second slit 172.

Certain embodiments of the present application relate to a needle 100 defining a lumen 110, the needle 100 comprising: a proximal portion 120 defining a first port 112 of the lumen 110; and a distal portion 130 defining second port 113 of the lumen 110, wherein the lumen 110 extends between and connects the first port 112 and the second port 113, and wherein the distal portion 130 comprises: an open tip portion 140 defining the second port 113, wherein the open tip portion 140 comprises at least one sharpened tip 142 configured to facilitate insertion of the distal portion 130 into patient tissue; an opening 164 connected with the lumen 110; and a ramp 162 at least partially obstructing the lumen 110 and configured to urge patient tissue within the distal portion 130 toward the opening 164 during insertion of the distal portion 130 into patient tissue.

In certain embodiments, the distal portion 130 further comprises at least one additional port 113 positioned proximally of the opening 164.

In certain embodiments, the distal portion 130 further comprises a plurality of slits 152 positioned proximally of the opening 164.

In certain embodiments, the plurality of slits 152 extend transverse to a longitudinal axis 131 of the distal portion 130.

In certain embodiments, the needle 100 comprises an outer surface 102 and an inner surface 104; and wherein the outer surface 102 is more hydrophilic than the inner surface 104.

In certain embodiments, at least one of the outer surface 102 or the inner surface 104 comprises a surface treatment 103, 105 that alters a hydrophilicity of the at least one of the outer surface 102 or the inner surface 104.

In certain embodiments, the distal portion 130 comprises a tab 160 that is deformed inward to thereby define the ramp 162 and the opening 164.

Certain embodiments of the present application relate to a flexible needle 100 defining a lumen 110, the needle 100 comprising: a proximal portion 120 defining a first port 112 of the lumen 110; and a distal portion 130 defining a second port 113 of the lumen 110, wherein the lumen 110 extends between and connects the first port 112 and the second port 113, and wherein the distal portion 130 comprises a tip portion 140 comprising at least one sharpened tip 142 configured to facilitate insertion of the distal portion 130 into patient tissue; wherein the flexible needle 100 is formed of nitinol; and wherein the nitinol is at least 95% austenitic at a temperature of 0° Celsius or lower.

In certain embodiments, the nitinol is configured to remain at least 95% austenitic when cooled from a temperature at which the nitinol is fully austenitic to the temperature of 0° Celsius or lower.

In certain embodiments, the a martensite start temperature of the nitinol is 0° Celsius or lower.

In certain embodiments, the nitinol is at least 95% austenitic at a temperature of −10° Celsius or lower.

In certain embodiments, the nitinol is at least 95% austenitic at a temperature of −20° Celsius or lower.

In certain embodiments, an outer surface 102 of the distal portion 130 has a greater hydrophilicity than an inner surface 104 of the distal portion 130.

In certain embodiments, an atomic ratio of nickel to titanium for the nitinol is between 1.01 and 1.05.

In certain embodiments, the atomic ratio of nickel to titanium for the nitinol is between 1.02 and 1.04.

In certain embodiments, the distal portion 130 further comprises a flexible region 150 comprising a plurality of slits 152.

In certain embodiments, the tip portion 140 comprises a plurality of the sharpened tips 142; and wherein the plurality of sharpened tips 142 are spaced from one another about a longitudinal axis 141 of the tip portion 140.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. A flexible needle defining a lumen, the flexible needle comprising:
a proximal portion defining a first port of the lumen; and
a distal portion defining at least one second port of the lumen, wherein the lumen extends between and connects the first port and the at least one second port, and wherein the distal portion comprises:
a tip portion comprising at least one sharpened tip configured to facilitate insertion of the distal portion into patient tissue; and
a compressible region comprising at least one slit, each slit defining a corresponding second port of the at least one second port;
wherein the compressible region is configured to compress during insertion of the distal portion into patient tissue to thereby close one or more second ports of the at least one second port, and to thereafter expand to open the one or more second ports.

2. The flexible needle of claim 1, wherein one or more slits of the at least one slit extends transverse to a longitudinal axis of the distal portion.

3. The flexible needle of claim 1, wherein the at least one second port comprises a plurality of second ports; and
wherein the tip portion further defines an additional second port of the plurality of second ports.

4. The flexible needle of claim 1, wherein the at least one sharpened tip comprises a plurality of sharpened tips.

5. The flexible needle of claim 4, wherein the plurality of sharpened tips are evenly spaced about a longitudinal axis of the tip portion.

6. The flexible needle of claim 1 wherein the compressible region is more flexible in a first direction transverse to a longitudinal axis of the distal portion and is less flexible in a second direction transverse to the longitudinal axis of the distal portion.

7. The flexible needle of claim 1, wherein the proximal portion further comprises:
a second compressible region comprising at least one second slit; and
a sheath covering the second compressible region to prevent fluid from exiting the at least one second slit.

8. A needle defining a lumen, the needle comprising:
a proximal portion defining a first port of the lumen; and
a distal portion defining second port of the lumen, wherein the lumen extends between and connects the first port and the second port, and wherein the distal portion comprises:
an open tip portion defining the second port, wherein the open tip portion comprises at least one sharpened tip configured to facilitate insertion of the distal portion into patient tissue;
an opening connected with the lumen; and
a ramp at least partially obstructing the lumen and configured to urge patient tissue within the distal portion toward the opening during insertion of the distal portion into patient tissue.

9. The needle of claim 8, wherein the distal portion further comprises at least one additional port positioned proximally of the opening.

10. The needle of claim 8, wherein the distal portion further comprises a plurality of slits positioned proximally of the opening.

11. The needle of claim 10, wherein the plurality of slits extend transverse to a longitudinal axis of the distal portion.

12. The needle of claim 8, wherein the needle comprises an outer surface and an inner surface; and
wherein the outer surface is more hydrophilic than the inner surface.

13. The needle of claim 12, wherein at least one of the outer surface or the inner surface comprises a surface treatment that alters a hydrophilicity of the at least one of the outer surface or the inner surface.

14. The needle of any of claim 8, wherein the distal portion comprises a tab that is deformed inward to thereby define the ramp and the opening.

15. A flexible needle defining a lumen, the needle comprising:
a proximal portion defining a first port of the lumen; and
a distal portion defining a second port of the lumen, wherein the lumen extends between and connects the first port and the second port, and wherein the distal portion comprises a tip portion comprising at least one sharpened tip configured to facilitate insertion of the distal portion into patient tissue;
wherein the flexible needle is formed of nitinol; and
wherein the nitinol is at least 95% austenitic at a temperature of 0° Celsius or lower.

16. The flexible needle of claim 15, wherein the nitinol is configured to remain at least 95% austenitic when cooled from a temperature at which the nitinol is fully austenitic to the temperature of 0° Celsius or lower.

17-19. (canceled)

20. The flexible needle of claim 15, wherein an outer surface of the distal portion has a greater hydrophilicity than an inner surface of the distal portion.

21. The flexible needle of claim 15, wherein an atomic ratio of nickel to titanium for the nitinol is between 1.01 and 1.05.

22. (canceled)

23. The flexible needle of claim 15, wherein the distal portion further comprises a flexible region comprising a plurality of slits.

24. The flexible needle of claim 15, wherein the tip portion comprises a plurality of the sharpened tips; and
wherein the plurality of sharpened tips are spaced from one another about a longitudinal axis of the tip portion.
